(19) United States
(12) Patent Application Publication
(10) Pub. No.: US 2026/0202294 A1
(43) Pub. Date: Jul. 16, 2026

(54) TESTING DEVICE FOR ENTIRE PROCESS OF BOREHOLE DEFORMATION-CRACK EVOLUTION OF COAL MASS AND EVALUATION METHOD THEREOF

(71) Applicants: Shandong University of Science and Technology, Qingdao (CN); SHANDONG ENERGY GROUP CO., LTD., Jinan (CN)

(72) Inventors: Yanchun YIN, Qingdao (CN); Shihang LI, Qingdao (CN); Yang CHEN, Qingdao (CN); Fangrui LIU, Qingdao (CN); Xinrong ZHANG, Qingdao (CN); Zitong GAO, Qingdao (CN)

(21) Appl. No.: 19/562,656

(22) Filed: Mar. 10, 2026

Related U.S. Application Data
(63) Continuation of application No. PCT/CN2024/075725, filed on Feb. 4, 2024.

(30) Foreign Application Priority Data
Nov. 15, 2023 (CN) 202311521482.0

(51) Int. Cl. G01N 3/12; E21B 49/00; G01N 3/06; G01N 33/22

(52) U.S. Cl. CPC G01N 3/12; E21B 49/006; G01N 3/068; G01N 33/222; G01N 2203/0003; G01N 2203/0048; G01N 2203/0066; G01N 2203/0218; G01N 2203/027; G01N 2203/0298; G01N 2203/0458; G01N 2203/0647; G01N 2203/0658

(57) ABSTRACT

The present disclosure provides a testing device and evaluation method for the entire process of borehole deformation-crack evolution of coal mass, which relates to the technical field of coal borehole deformation testing. The testing device includes a loading mechanism and a monitoring mechanism; the loading mechanism includes an outer frame, a bearing plate, a loading cylinder, a spacer block, and a coal specimen; the monitoring mechanism includes an industrial camera, an acoustic emission detector, and an information acquisition unit. This testing device uses high-strength transparent material bearing plate to achieve visual observation; for monitoring the status of borehole, a borehole deformation monitoring sensor or a miniature camera can be selected to achieve the entire process testing of borehole from deformation to closure.

### CROSS-REFERENCE TO RELATED APPLICATIONS

This application is a continuation of International Application No. PCT/CN2024/075725 with a filling date of Feb. 4, 2024, designating the United states, now pending, and further claims to the benefit of priority from Chinese Application No. 202311521482.0 with a filing date of Nov. 15, 2023. The content of the aforementioned applications, including any intervening amendments thereto, are incorporated herein by reference.

### TECHNICAL FIELD

The present disclosure relates to the technical field of coal borehole deformation testing, in particular to a testing device for the entire process of borehole deformation-crack evolution of coal mass and an evaluation method thereof.

### BACKGROUND

Rock burst is a typical mining dynamic phenomenon that can release a large amount of elastic deformation energy accumulated in the coal rock mass in a rapid and violent manner, causing damage to the coal rock mass and generating strong vibrations. The dynamic force throws the broken coal rock into the mining space of the roadway, making a strong noise, causing equipment damage, roadway damage, and personnel casualties. In the prevention and control of rock burst, large-diameter borehole pressure relief is one of the main methods, which has the characteristics of low construction difficulty and obvious pressure relief effect. Therefore, this technology is widely used.

Large-diameter borehole pressure relief is a method of reducing the stress concentration of nearby coal or changing the mechanical properties of nearby coal through the construction of large-diameter borehole, in order to eliminate or reduce the risk of deformation and damage to the surrounding rock of the roadway. The pressure relief effect of drilling borehole is closely related to the borehole deformation and the range of coal fracture around the borehole. With the deformation, shrinkage, and collapse of borehole, the cracks in the coal around the borehole continue to expand, and the pressure relief effect gradually becomes effective. Therefore, monitoring the deformation of boreholes and surrounding coal mass is helpful for studying the pressure relief effect of boreholes and effectively guiding the design of pressure relief parameters.

However, at present, research on the pressure relief effect of coal mass containing boreholes mainly focuses on the overall mechanical properties and impact tendency of the coal mass, with little attention paid to the deformation process of the boreholes themselves. There is a lack of effective monitoring and quantitative evaluation methods for borehole deformation in current. Therefore, the present disclosure proposes a testing device and evaluation method for the entire process of borehole deformation-crack evolution of coal mass.

### SUMMARY

The objective of the present disclosure is to provide a testing device and evaluation method for the entire process of borehole deformation-crack evolution of coal mass, which can monitor the deformation of boreholes and the micro fracture information of nearby coal mass while loading coal specimens containing boreholes.

In order to achieve the above objective, the technical solution adopted by the present disclosure is as follows:

A testing device for entire process of borehole deformation-crack evolution of coal mass includes a loading mechanism and a monitoring mechanism, wherein the loading mechanism includes an outer frame, a bearing plate, a loading cylinder, a spacer block, and a coal specimen; one end of the outer frame is fixedly provided with the bearing plate, and a through hole is opened at the center position of the bearing plate; the coal specimen is set between the outer frame and the bearing plate; and the coal specimen is provided with a borehole at the corresponding the through hole. Except for the side connected to the bearing plate and the opposite side, the coal specimen is in contact with the spacer block. The loading cylinder is installed on the spacer block, and the spacer block is provided with holes.

The monitoring mechanism includes an industrial camera, an acoustic emission detector, and an information acquisition unit, wherein the industrial camera is placed in front of the bearing plate and opposite to the through hole; the acoustic emission detector is placed in the hole opened in the spacer block and arranged on a surface of the coal specimen; and the information acquisition unit is configured to monitor and obtain status information of the borehole.

Preferably, the information acquisition unit is set as a miniature camera, and the miniature camera is set in the through hole opened in the bearing plate.

Preferably, the information acquisition unit is set as a borehole deformation monitoring sensor, wherein the borehole deformation monitoring sensor includes a water bag, a conduit, and a flow monitor; the water bag is placed inside the borehole of the coal specimen, and the flow monitor is connected to the water bag through the conduit.

Preferably, the outer frame includes a first support plate, a second support plate, and a plurality of support rods, wherein the first support plate and the second support plate are arranged in parallel, and four corner positions of the first support plate are correspondingly connected to four corner positions of the second support plate through the plurality of support rods; the first support plate is set at one end of each support rod, and the second support plate is set at a middle position of each support rod; an other end of each support rod is fixedly connected to the bearing plate, and the coal specimen is set between the bearing plate and the second support plate.

Preferably, the spacer block is provided with a hole arranged along a diagonal line of the spacer block.

Preferably, the bearing plate is made of high-strength transparent material.

Further, the present disclosure provides an evaluation method for a testing device for the entire process of borehole deformation-crack evolution of coal mass, using the testing device for the entire process of borehole deformation-crack evolution of coal mass mentioned above, which includes the following steps:
Step 1: selecting coal of various strengths as test samples, preparing a plurality of cubic coal specimens, drilling a borehole in a middle of the coal specimen in advance to obtain coal specimen containing the borehole, and creating an artificial speckle field on a surface where the coal specimen are drilled;
Step 2: installing the coal specimen, placing the spacer block between the coal specimen and the loading cylinder, placing the acoustic emission detector in the hole opened in the spacer block, and arranging the acoustic emission detector on the surface of the coal specimen;
Step 3: placing a industrial camera in front of a transparent bearing plate, and the industrial camera directly faces the through hole of the bearing plate;
Step 4: for monitoring borehole status, the first or second method is used for comprehensive analysis:
Method 1: installing a miniature camera in the through hole of the bearing plate at the front end of the borehole to capture the shape of the internal borehole wall;
Method 2: Placing the water bag of the borehole deformation monitoring sensor in the borehole, and connecting the flow monitor to the water bag through the conduit to monitor and obtain information on the entire deformation-closure process of the borehole;
Step 5: for coal loading scheme: a horizontal stress of the coal specimen is σ2=σ3, and the horizontal stress remains unchanged during a loading process; setting multiple sets of stress values in sequence; a vertical stress of the specimen σ1 is loaded using displacement control, setting the loading speed; during a test, after synchronously loading σ1, σ2 and σ3 to a set horizontal stress value, σ2 and σ3 remain unchanged, and σ1 continues to be loaded;
Step 6: switching on the miniature camera or the borehole deformation monitoring sensor for data acquisition, loading through a loading cylinder until the borehole is closed, and continuing to load 2 mm before stopping the test;
Step 7: performing quantitative analysis of coal fracture and deformation around the borehole based on monitoring data:
The overall rupture degree:

$$a = \frac{{n}_{AE}^{i}}{{n}_{AE}} ,$$

Wherein,

$${n}_{AE}^{i}$$

is the cumulative ringing number at a certain moment i, and n_{AE} is the cumulative ringing number of acoustic emission when the test is completed;
The crack propagation index:

$$β = {an}_{AE}^{{R}_{1}} + {bn}_{AE}^{{R}_{2}} + {cn}_{AE}^{{R}_{3}} + {dn}_{AE}^{{R}_{4}} ,$$

Wherein, a, b, c and d are weight coefficients,

$${n}_{AE}^{{R}_{1}} , {n}_{AE}^{{R}_{2}} , {n}_{AE}^{{R}_{3}} , {n}_{AE}^{{R}_{4}}$$

are the number of acoustic emission events within different radius ranges from a center of the borehole;
The borehole closure:

$$S = \frac{{A}_{i}}{{A}_{initial}} ,$$

Wherein, Ai is a borehole area at a certain moment, Aᵢₙᵢₜᵢₐₗ is a borehole area at an initial moment;
Or

$$S = \frac{{V}_{i}}{{V}_{total}} ,$$

Wherein, Vi is cumulative drainage volume at a certain moment i, and Vₜₒₜₐₗ is total water volume in the water bag.

Advantageous technical effects of the present disclosure are shown as below:

The present disclosure relates to a testing device and evaluation method for the entire process of borehole deformation-crack evolution of coal mass. By using a high-strength transparent material bearing plate, the visualization observation of the pressure-bearing surface containing the borehole under triaxial stress loading is achieved, which facilitates intuitive or monitoring of the deformation of the borehole and the micro fracture information of the nearby coal mass using a monitoring device; by monitoring the borehole status, it is possible to choose to use borehole deformation monitoring sensors or miniature cameras, providing diversified methods for entire process monitoring, with more comprehensive monitoring information, and realizing the full process testing of borehole from deformation to closure; by quantitatively characterizing the deformation of the borehole, a quantitative evaluation of the borehole deformation has been achieved from multiple perspectives, including overall rupture, crack propagation around the borehole, and shrinkage deformation of the borehole.

### BRIEF DESCRIPTION OF THE DRAWINGS

FIG. 1 is a three-dimensional schematic diagram of a testing device in an embodiment of the present disclosure;

FIG. 2 is a three-dimensional schematic diagram of the outer frame in the embodiment of the present disclosure;

FIG. 3 is the assembly diagram of a coal specimen, a spacer block, an acoustic emission detector and a loading cylinder in the embodiment of the disclosure;

FIG. 4 is the structural diagram of the coal specimen and the acoustic emission detector in the embodiment of the disclosure;

FIG. 5 is a schematic diagram of the structure of a borehole deformation monitoring sensor in the embodiment of the present disclosure;

FIG. 6 is the assembly diagram of the coal specimen and the borehole deformation monitoring sensor in the embodiment of the disclosure;

FIG. 7 is the sectional view of the coal specimen, the miniature camera and the bearing plate in the embodiment of the disclosure;

FIG. 8 shows the stress loading path of coal mass containing boreholes in the embodiment of the present disclosure.

Wherein the reference numbers in the drawings: 1-loading mechanism: 11-outer frame, 111-first support plate, 112-second support plate, 113-support rod;
12-loading cylinder, 13-bearing plate, 131-through-hole; 14-spacer block; 15-coal specimen, 151-borehole;
2-monitoring mechanism: 21-industrial camera, 22-acoustic emission detector, 23-miniature camera;
24-borehole deformation monitoring sensor, 241-water bag, 242-conduit, 243-flow monitor.

### DETAILED DESCRIPTION OF THE EMBODIMENT

The present invention will be further described with reference to the drawings and preferred embodiments. It should be understood that these embodiments are only used to illustrate the present invention, but the present invention is not limited thereto.

In the present invention, the terms “first,” “second,” and “third” are merely for the purpose of description, but cannot be understood as indicating or implying relative importance. The term “multiple” means two or more unless otherwise explicitly defined. The terms “mount,” “connect with,” “connect,” “fix,” and the like shall be understood in a broad sense. For example, “connect” may mean being fixedly connected, detachably connected, or integrally connected; and “connect with” may mean being directly connected or indirectly connected through an intermediary. For those of ordinary skill in the art, specific meanings of the above terms in the present invention can be understood according to specific situations.

In the description of the present invention, it should be understood that if orientation or position relations indicated by the terms such as “upper,” “lower,” “left,” “right,” “front,” “back,” and the like are based on the orientation or position relations shown in the drawings, and the terms are intended only to facilitate the description of the present invention and simplify the description, rather than indicating or implying that the apparatus or element referred to must have a particular orientation and be constructed and operated in the particular orientation, and therefore cannot be construed as a limitation on the present invention.

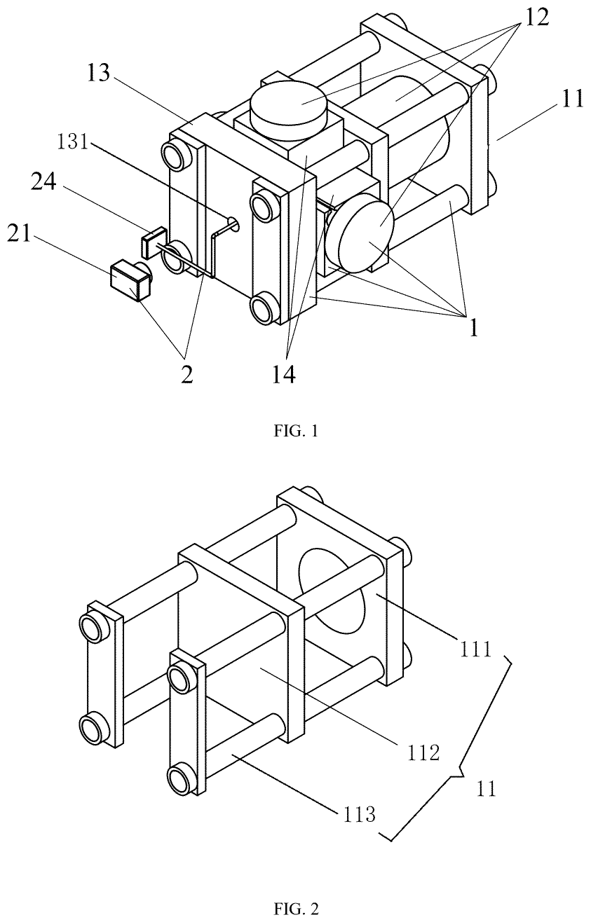

The present disclosure involves a testing device for entire process of borehole deformation-crack evolution of coal mass, which includes a loading mechanism 1 and a monitoring mechanism 2. As shown in FIG. 1-FIG. 4, the loading mechanism 1 includes structural components such as an outer frame 11, a loading cylinder 12, a bearing plate 13, a spacer block 14, and a coal specimen 15.

As shown in FIG. 2, the outer frame 11 includes a first support plate 111, a second support plate 112, and a plurality of support rods 113. The first support plate 111 and the second support plate 112 are arranged in parallel, and the four corner positions of the first support plate 111 are correspondingly connected to the four corner positions of the second support plate 112 through the plurality of support rods 113. The first support plate 111 is set at one end of each support rod 113, and the second support plate 112 is set at the middle position of each support rod 112. As shown in FIG. 1, the other end of each support rod 113 is fixedly connected to the bearing plate 13, The center position of the bearing plate 13 is provided with a through hole 131, and the bearing plate 13 is made of high-strength transparent material. The bearing plate 13 is used to limit the displacement of the coal specimen 15 and facilitate the use of an industrial camera 21 to take speckle images. The coal specimen 15 is set between the bearing plate 13 and the second support plate 112. By using the high-strength transparent material bearing plate 13, visual observation of the pressure-bearing surface containing the borehole under triaxial stress loading is achieved, which facilitates intuitive or monitoring of the deformation of the borehole and micro fracture information of nearby coal mass using monitoring devices.

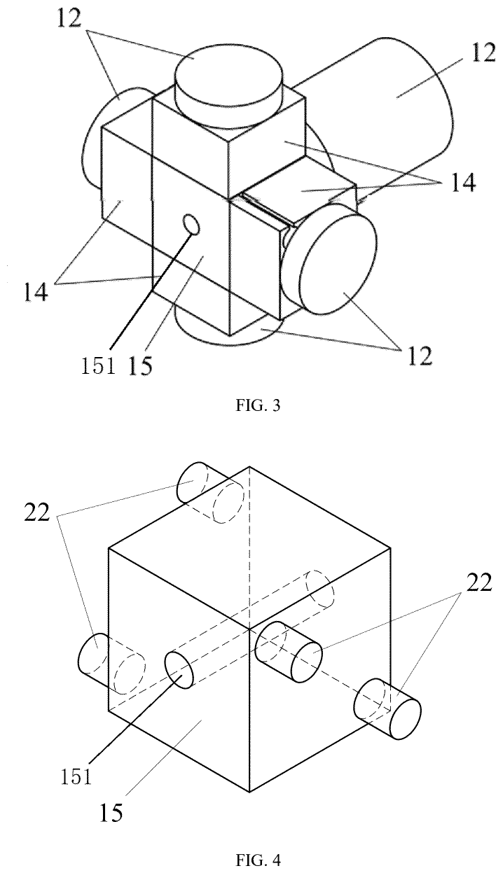

As shown in FIG. 3, a borehole 151 is drilled at the position of the coal specimen 15 corresponding to the through hole 131. Except for the side connected to the bearing plate 13 and the side opposite to the side connected to the bearing plate 13, the coal specimen 15 is in contact with the spacer block 14. The spacer block 14 is equipped with a loading cylinder 12. The position of the loading cylinder 12 is adjusted to adapt to the loading test of coal specimens 15 of different sizes, and the loading cylinder 12 is controlled to apply load to the coal specimen 15. As shown in FIG. 4, there are holes on the spacer block 14 that can be used to install the acoustic emission detector 22, and the holes are arranged along the diagonal of the spacer block 14.

As shown in FIG. 4, the monitoring mechanism 2 includes an industrial camera 21, an acoustic emission detector 22, and an information acquisition unit. The industrial camera 21 is placed in front of the bearing plate 13 and opposite to the through hole 131 opened in the center of the bearing plate 1. An artificial speckle field is created on the side of the coal specimen 15 connected to the bearing plate 13, the industrial camera 21 captures speckle images of the entire test process, and the deformation field of the surface of the coal specimen 15 is obtained using digital image correlation method (DIC), and the data on the shape and surface area of the borehole 151 are obtained using digital image processing technology (DIP). The acoustic emission detector 22 is placed in the hole opened in the spacer block 14 and arranged on the surface of the coal specimen 15. By using the acoustic emission detector 22 to monitor the micro fracture information of the coal mass near the borehole 151.

The information acquisition unit is configured to monitor and obtain status information of the borehole 151.

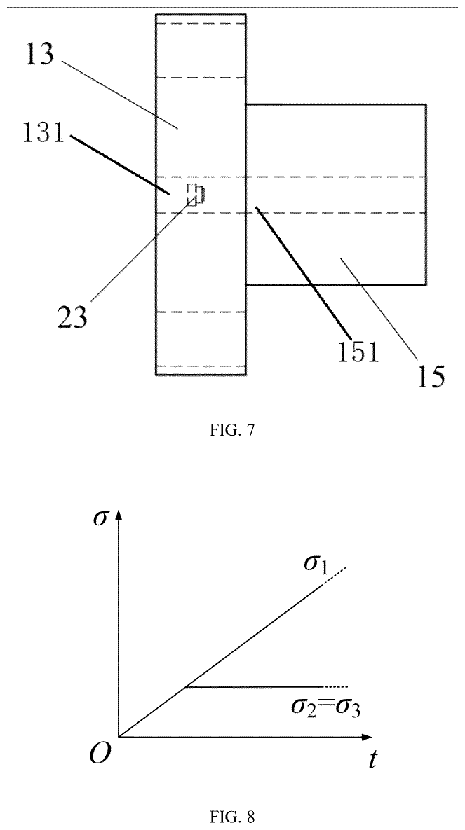

As shown in FIG. 7, the information acquisition unit is set as a miniature camera 23, and the miniature camera 23 is set in the through hole 131 opened in the bearing plate 13. The miniature camera 23 is capable of capturing the shape of the inner wall of borehole 151.

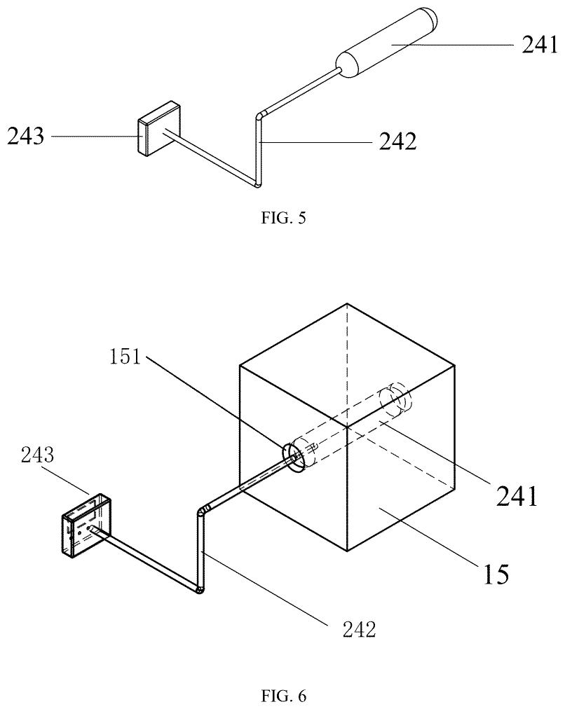

As shown in FIG. 5 and FIG. 6, the information acquisition unit is set as a borehole deformation monitoring sensor 24. The borehole deformation monitoring sensor 24 includes a water bag 241, a conduit 242, and a flow monitor 243. The water bag 241 is placed inside the borehole 151 of the coal specimen 15, and the flow monitor 243 is connected to the water bag 241 through the conduit 242. The borehole deformation monitoring sensor 24 is used to monitor and obtain the overall deformation-closure process information of the borehole 151.

This device can choose to use the borehole deformation monitoring sensor 24 or the miniature camera 23 to monitor the status of borehole 151, providing diversified methods for full process monitoring and more comprehensive monitoring information, achieving the full process testing of borehole 151 from deformation to closure.

Referring to FIG. 1-FIG. 8, the present disclosure provides an evaluation method for a testing device for the entire process of borehole deformation-crack evolution of coal mass, using the testing device for the entire process of borehole deformation-crack evolution of coal mass mentioned above, which includes the following steps:
Step 1: selecting 3-4 types of coal with different strengths as test samples, preparing a plurality of cubic coal specimens 15 with the size of 100×100×100 mm, respectively drilling boreholes with bore diameter of 8, 10, 12, 14, 16 mm in the middle of the coal specimens 15 in advance to obtain coal specimens containing boreholes, and creating an artificial speckle field on a surface where the coal specimen is connected to the bearing plate 13;
Step 2: installing one of the coal specimens 15 each time for testing, placing the spacer block 14 between the coal specimen 15 and the loading cylinder 12, placing the acoustic emission detector 22 in the hole opened in the spacer block 14, and arranging the acoustic emission detector 22 on the surface of the coal specimen 15;
Step 3: placing a industrial camera 21 in front of a transparent bearing plate 13, and the industrial camera 21 directly faces the through hole 131 of the bearing plate 13;
Step 4: for monitoring borehole status, the first or second method is used for comprehensive analysis:
Method 1: installing a miniature camera 23 in the through hole 131 of the bearing plate 13 at the front end of the borehole 151 to capture the shape of the internal wall of the borehole 151;
Method 2: Placing the water bag 241 of the borehole deformation monitoring sensor 241 in the borehole, and connecting the flow monitor 243 to the water bag 241 through the conduit 242 to monitor and obtain information on the entire deformation-closure process of the borehole 151;
Step 5: for coal loading scheme: the horizontal stress of the coal specimen is σ2=σ3, and the horizontal stress remains unchanged during a loading process; setting multiple sets of stress values as 50%, 80%, 100%, 120% and 150% of σc (uniaxial compression strength); the vertical stress of the specimen σ1 is loaded using displacement control, setting the loading speed as 0.01, 0.02, 0.04, 0.06, 0.08 and 0.1 mm/min; during a test, after synchronously loading σ1, σ2 and σ3 to the set horizontal stress value, σ2 and σ3 remain unchanged, and σ1 continues to be loaded;
Step 6: switching on the miniature camera 23 or the borehole deformation monitoring sensor 24 for data acquisition, loading through a loading cylinder 12 until the borehole 151 is closed, and continuing to load 2 mm before stopping the test;
Step 7: performing quantitative analysis of coal fracture and deformation around the borehole 151 based on monitoring data:
The overall rupture degree:

$$a = \frac{{n}_{AE}^{i}}{{n}_{AE}} ,$$

Wherein,

$${n}_{AE}^{i}$$

is the cumulative ringing number at a certain moment i, and n_{AE} is the cumulative ringing number of acoustic emission when the test is completed;
The crack propagation index:

$$β = {an}_{AE}^{{R}_{1}} + {bn}_{AE}^{{R}_{2}} + {cn}_{AE}^{{R}_{3}} + {dn}_{AE}^{{R}_{4}} ,$$

Wherein, a, b, c and d are weight coefficients,

$${n}_{AE}^{{R}_{1}} , {n}_{AE}^{{R}_{2}} , {n}_{AE}^{{R}_{3}} , {n}_{AE}^{{R}_{4}}$$

are the number of acoustic emission events within different radius ranges from a center of the borehole;
The borehole closure:

$$S = \frac{{A}_{i}}{{A}_{initial}} ,$$

Wherein, Ai is a borehole area at a certain moment, Aᵢₙᵢₜᵢₐₗ is a borehole area at an initial moment;
Or

$$S = \frac{{V}_{i}}{{V}_{total}} ,$$

Wherein, Vi is cumulative drainage volume at a certain moment i, and Vₜₒₜₐₗ is total water volume in the water bag 241.

Thus, a detailed description of this embodiment has been provided in conjunction with the accompanying drawings. Based on the above description, skilled person in the art should have a clear understanding of the testing device and evaluation method for the entire process of borehole deformation-crack evolution of coal mass provided by the present invention. By quantitatively characterizing the deformation of the borehole, a quantitative evaluation of the borehole deformation has been achieved from multiple perspectives, including overall rupture, propagation of cracks around the borehole, and shrinkage deformation of the borehole.

Certainly, the above descriptions are merely preferred embodiments of the present disclosure. The present disclosure is not limited to the above embodiments listed. It should be noted that, all equivalent replacements and obvious variations made by any person skilled in the art under the teaching of the specification fall within the essential scope of the specification and shall be protected by the present disclosure.

What is claimed is:

1. A testing device for entire process of borehole deformation-crack evolution of coal mass, comprising a loading mechanism and a monitoring mechanism;
wherein the loading mechanism comprises an outer frame, a bearing plate, a loading cylinder, a spacer block, and a coal specimen;
the outer frame comprises a first support plate, a second support plate, and a plurality of support rods;
the first support plate and the second support plate are arranged in parallel, and four corner positions of the first support plate are correspondingly connected to four corner positions of the second support plate through the plurality of support rods; the first support plate is set at one end of each support rod, and the second support plate is set at a middle position of each support rod; an other end of each support rod is fixedly connected to the bearing plate, and the coal specimen is set between the bearing plate and the second support plate;
a center position of the bearing plate is provided with a through hole, the coal specimen is provided with a borehole corresponding to the through hole, the coal specimen is in contact with the spacer block except for a side connected to the bearing plate and a side opposite to the side connected to the bearing plate, the loading cylinder is arranged on the spacer block, and the spacer block is provided with a hole arranged along a diagonal line of the spacer block;
the monitoring mechanism comprises an industrial camera, an acoustic emission detector, and an information acquisition unit;
the industrial camera is placed in front of the bearing plate and opposite to the through hole; the acoustic emission detector is placed in the hole opened in the spacer block and arranged on a surface of the coal specimen; the information acquisition unit is configured to monitor and obtain status information of the borehole, and is set as a borehole deformation monitoring sensor;
the borehole deformation monitoring sensor comprises a water bag, a conduit, and a flow monitor; the water bag is placed inside the borehole of the coal specimen, and the flow monitor is connected to the water bag through the conduit.

2. The testing device for the entire process of borehole deformation-crack evolution of coal mass according to claim 1, wherein the bearing plate is made of high-strength transparent material.

3. An evaluation method for a testing device for the entire process of borehole deformation-crack evolution of coal mass, using the testing device for the entire process of borehole deformation-crack evolution of coal mass as claimed in claim 1, comprising the following steps:
step 1: selecting coal of various strengths as test samples, preparing a plurality of cubic coal specimens, drilling a borehole in a middle of the coal specimen in advance to obtain coal specimen containing the borehole, and creating an artificial speckle field on a surface where the coal specimen are drilled;
step 2: installing the coal specimen, placing the spacer block between the coal specimen and the loading cylinder, placing the acoustic emission detector in the hole opened in the spacer block, and arranging the acoustic emission detector on the surface of the coal specimen;
step 3: placing a industrial camera in front of a transparent bearing plate, and the industrial camera directly faces the through hole of the bearing plate;
step 4: for monitoring borehole status, a following method is used for comprehensive analysis:
placing a water bag of a borehole deformation monitoring sensor in the borehole, and connecting a flow monitor to the water bag through a conduit to monitor and obtain information on an entire process of deformation-closure process of the borehole;
step 5: for coal loading scheme: a horizontal stress of the coal specimen is σ2=σ3, and the horizontal stress remains unchanged during a loading process; setting multiple sets of stress values in sequence; a vertical stress of the specimen σ1 is loaded using displacement control, setting the loading speed; during a test, after synchronously loading σ1, σ2 and σ3 to a set horizontal stress value, σ2 and σ3 remain unchanged, and σ1 continues to be loaded;
step 6: switching on the borehole deformation monitoring sensor for data acquisition, loading through a loading cylinder until the borehole is closed, and continuing to load 2 mm before stopping the test;
step 7: performing quantitative analysis of coal fracture and deformation around the borehole based on monitoring data:
an overall rupture degree:
$$a = \frac{{n}_{A E}^{i}}{{n}_{A E}} ,$$
wherein,
$${n}_{A E}^{i}$$
is the cumulative ringing number at a certain moment i, and n_{AE} is the cumulative ringing number of acoustic emission when the test is completed;
the crack propagation index:
$$β = a {n}_{A E}^{{R}_{1}} + b {n}_{A E}^{{R}_{2}} + c {n}_{A E}^{{R}_{3}} + d {n}_{A E}^{{R}_{4}} ,$$
wherein, a, b, c and d are weight coefficients,
$${n}_{AE}^{{R}_{1}} , {n}_{AE}^{{R}_{2}} , {n}_{AE}^{{R}_{3}} , {n}_{AE}^{{R}_{4}}$$
are the number of acoustic emission events within different radius ranges from a center of the borehole;
the borehole closure:
$$S = \frac{{A}_{i}}{{A}_{initial}} ,$$
wherein, Ai is a borehole area at a certain moment, Aᵢₙᵢₜᵢₐₗ is a borehole area at an initial moment;
or
$$S = \frac{{V}_{i}}{{V}_{total}} ,$$
wherein, Vi is cumulative drainage volume at a certain moment i, and Vₜₒₜₐₗ is total water volume in the water bag.
